(19) United States
(12) Patent Application Publication
(10) Pub. No.: US 2021/0238537 A1
(43) Pub. Date: Aug. 5, 2021

(54) CONDUCTIVITY-BASED LYSIS MONITORS

(71) Applicant: HEWLETT-PACKARD DEVELOPMENT COMPANY, L.P., Spring, TX (US)

(72) Inventors: Alexander Govyadinov, Corvallis, OR (US); Diane R. Hammerstad, Corvallis, OR (US); Viktor Shkolnikov, Palo Alto, CA (US)

(73) Assignee: HEWLETT-PACKARD DEVELOPMENT COMPANY, L.P., Spring, TX (US)

(21) Appl. No.: 17/049,406

(22) PCT Filed: Aug. 10, 2018
(86) PCT No.: PCT/US2018/046201

§ 371 Date: Oct. 21, 2020

(51) Int. Cl. C12M 1/00; C12M 1/36; C12N 1/06

(52) U.S. Cl. CPC C12M 47/06; C12N 1/066; C12M 41/48

(57) ABSTRACT

In one example in accordance with the present disclosure, a conductivity-based lysis monitor is described. The lysis monitoring device includes a lysing chamber to receive a cell to be lysed and at least one lysing device to rupture a cell membrane. At least one pair of electrodes are disposed in the lysing chamber to detect a level of conductivity in the lysing chamber. A controller of the device determines when the cell membrane has ruptured based on detected levels of conductivity in the lysing chamber.

### BACKGROUND

Cell lysis is a process of rupturing the cell membrane to extract intracellular components for purposes such as purifying the components, retrieving deoxyribonucleic acid (DNA), ribonucleic acid (RNA), proteins, polypeptides, metabolites, or other small molecules contained therein, and analyzing the components for genetic and/or disease characteristics. Cell lysis bursts a cell's membrane and frees the cell's inner components. The fluid containing the cell's inner components is referred to as lysate.

### BRIEF DESCRIPTION OF THE DRAWINGS

The accompanying drawings illustrate various examples of the principles described herein and are part of the specification. The illustrated examples are given merely for illustration, and do not limit the scope of the claims.

FIG. 1 is a block diagram of a conductivity-based lysis monitoring device, according to an example of the principles described herein.

FIG. 2 is a diagram of a conductivity-based cell lysis system, according to an example of the principles described herein.

FIG. 3 is a flowchart of a method for conductivity-based lysis monitoring, according to an example of the principles described herein.

FIGS. 4A-4D are diagrams of conductivity-based lysis monitoring, according to an example of the principles described herein.

FIG. 5 is a flowchart of a method for conductivity-based lysis monitoring, according to another example of the principles described herein.

FIG. 6 is a diagram of a conductivity-based cell lysis system, according to another example of the principles described herein.

FIG. 7 is a diagram of a conductivity-based cell lysis system, according to another example of the principles described herein.

FIG. 8 is a diagram of a conductivity-based cell lysis system, according to another example of the principles described herein.

FIG. 9 is a diagram of a conductivity-based cell lysis system, according to another example of the principles described herein.

FIG. 10 is a diagram of a conductivity-based cell lysis system, according to another example of the principles described herein.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

Cell lysis is a process of extracting intracellular components for purposes such as purifying the components, retrieving DNA and RNA proteins, polypeptides, metabolites, and small molecules or other components therein, and analyzing the components for genetic and/or disease characteristics. Cell lysis ruptures a cell membrane and frees the inner components. The fluid containing the inner components is referred to as lysate. The contents of the cell can then be analyzed by a downstream system. Cell lysis can be executed using any number of methods.

In one example, high frequency sound waves shear the cell membranes, and in some cases the cell walls if present. Another example of lysis via shearing is to mill the cells against balls in a fluid. In yet another example, a pestle may be used to rupture the cell membranes. In still another example of shearing, rotating blades may grind the cell membranes. Other examples of lysis include localized heating which can cause cell denaturation and can cause certain cells to rupture. As yet another example, the cells may be forced through a narrow space, thereby shearing the cell membranes. In another example, repeated cycles of freezing and thawing can disrupt cells through ice crystal formation. Solution-based lysis is another example wherein contents of a cell are extracted. In these examples, the osmotic pressure in the cell could be increased or decreased to collapse the cell membrane or to cause the cell membrane to burst.

In one particular example, lysis is triggered via a thermal resistor disposed within a microfluidic channel. The thermal resistor generates a vapor bubble. The expansion and collapse of the vapor bubble both produce a high pressure spike within the channel. This high pressure spike and the high shear within this localized area lyses a cell or cells within the localized area. In a particular example, a method of cell lysis includes moving cell fluid from a first reservoir through a microfluidic channel toward a second reservoir. A lysing device is activated to lyse the cell. Lysate fluid resulting from the activation of the lysing device is then moved through the microfluidic channel into a second reservoir. While particular examples of cell lysis mechanisms have been described herein, a variety of cell lysis mechanisms are used in biochemical analytics.

As cell lysis is a step in many sample preparation protocols for the characterization of nucleic acid or protein contents of a cell, the quality of cell lysis can have a direct impact on downstream operations. For example, if the lysis has poor efficiency, the amount of material to be analyzed may be reduced. Poor lysis can also affect the analytic results as those cells that are not lysed are excluded from the analysis. On the other hand, if the lysis conditions are too harsh, the nucleic acid and/or protein material may deteriorate. Doing so similarly degrades the information that can be obtained from the sample.

To offset these potential issues, chemists may execute the lysis operation with excessive power or time to ensure a high enough rate of lysis completion. This may be too much for some cells and can lead to degradation of the biomaterials of interest. Moreover, using excessive power and/or time is ineffective as a chemist may not know the exact moment when lysis is completed to a satisfactory level. Accordingly, extra resources and time are expended in an attempt to ensure that lysing is complete.

In other examples, a chemist may use a predetermined amount of reagent and/or perform predefined procedures to the sample to lyse the cells. In these cases, it may be assumed that all the steps in the preparation process go as planned and just the final results, following the entire chemical analysis, are measured. However, such a system is ineffective and may be inaccurate as the procedural steps may not be executed as expected.

Accordingly, the present specification describes a device, system, and method for addressing these and other issues. Specifically, the present specification describes a device for monitoring and controlling individual cell lysis by detecting and analyzing a change of impedance of the solution. That is, as the cell is lysed and contents therein are expelled, the conductivity of a solution will change. Accordingly, the present device includes a small chamber (in some examples no more than 100× the volume of the cell) with electrodes disposed therein. The chamber also includes a lysing device such as a pinch region and/or a physical lysing device such as a thermal resistor.

Upon lysing, the content of the cell is released, increasing the total conductivity. Each cell releases approximately the same amount of ions in the solution and so a digital count can be performed of the number of cells that have been lysed. This allows a user to approximate the amount of starting material for subsequent operations. In some examples, the system may include pumps activated by the data from the electrodes to enable the return of the cell to the lysing chamber in the event of insufficient lysing. In other words, a cell may be passed through the lysing chamber multiple times to ensure satisfactory lysis.

Specifically, the present specification describes a lysis monitoring device. The lysis monitoring device includes a lysing chamber to receive a cell to be lysed and at least one lysing device to rupture a cell membrane. The lysis monitoring device includes at least one pair of electrodes disposed in the lysing chamber to detect a level of conductivity in the lysing chamber. A controller of the lysis monitoring device determines when the cell membranes has ruptured based on detected levels of conductivity in the lysing chamber.

The present specification also describes a cell lysis system. The cell lysis system includes a cell fluid inlet and at least one lysis monitoring device fluidly connected to the cell fluid inlet. Each lysis monitoring device includes 1) a lysing chamber, 2) a lysing device disposed at least partially in the lysing chamber to rupture a cell membranes, 3) at least one pair of electrodes disposed in the lysing chamber to detect a level of conductivity in the lysing chamber, and 4) a controller to perform at least one of 1) determine a presence of the cell to be lysed in the lysing chamber; 2) activate the lysing device in response to a determined presence of the cell; and 3) determine when the cell membrane has ruptured. In this example, each lysis monitoring device also includes a pump to generate a fluid flow through the lysing chamber. The cell lysis system also includes a lysate outlet to a lysate from the at least one lysis monitoring device.

The present specification also describes a method. According to the method, a cell to be lysed is received in a lysing chamber. A lysing device in the lysing chamber is activated to rupture a cell membrane. A conductivity within the lysing chamber is measured and analyzed to determine if the cell membrane has ruptured. If a cell is un-lysed, it is re-directed to the lysing chamber.

In summary, using such a lysis system 1) provides for effective monitoring of cell lysis; 2) ensures sufficient lysis without degradation to cell contents; 3) provides control of the amount of analyte to be delivered downstream; 4) identifies subsets of cell population that are difficult to lyse; and 5) provides a feedback signal for automated control of the lysis operation. However, the devices disclosed herein may address other matters and deficiencies in a number of technical areas.

As used in the present specification and in the appended claims the term “cell membrane” refers to any membrane, wall, or other enclosure of a cell.

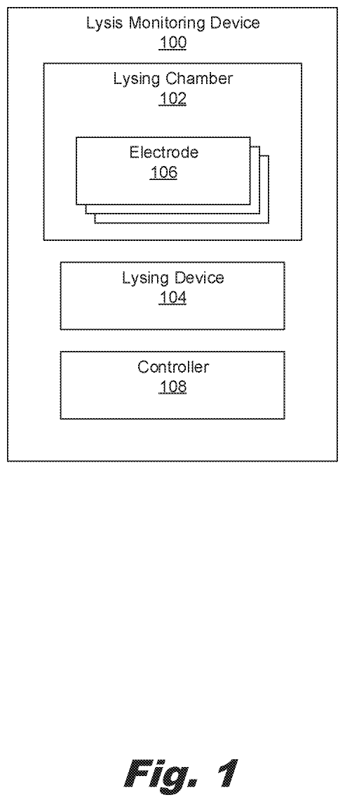

Turning now to the figures, FIG. 1 is a block diagram of a conductivity-based lysis monitoring device (100), according to an example of the principles described herein. As described above, the lysis monitoring device (100) provides a quality control check over a lysing operation. Specifically, the lysis monitoring device (100) includes a lysing chamber (102) where lysing occurs. In some examples, the lysing chamber (102) may be no more than 100 times a volume of a cell to be lysed. In other examples, the lysing chamber (102) may be of a size comparable with the cell and in some cases even smaller so as to deform the cell before or during the rupturing of the cell membrane. That is, the lysing chamber (102) may be a microfluidic structure. As the lysing chamber (102) is the location where lysis occurs, the lysing chamber (102) receives, through an input, a cell or other component to be lysed. In some examples as will be described below, the lysing chamber (102) may have a reduced or non-reduced cross-section relative to cell fluid inlets and lysate outlets.

The lysis monitoring device (100) also includes a lysing device (104) to rupture a cell membrane. That is, a cell has a wall or a membrane. It may be desirable to rupture that wall or membrane to expel the contents therein. Lysis refers to the operation of rupturing the cell wall or cell membrane and a lysing device (104) is a component of the lysis monitoring device (100) that carries out that operation. In some examples, the lysing device (104) is disposed outside of the lysing chamber (102) and in others, the lysing device (104) is disposed within the lysing chamber (102).

The lysing device (104) may take many forms. For example, lysing may occur simply by pushing the cells through a constriction. In this example, the lysing device (104) may be a transition between a wide input reservoir to a narrower lysing chamber (102). In other examples, the lysing device (104) may be a physical element. For example, the lysing device (104) may be a thermal inkjet resistor. That is, the lysing device (104) may include a firing resistor. In this example, the firing resistor heats up in response to an applied current. As the firing resistor heats up, a portion of the fluid in the lysing chamber (102) vaporizes to generate a bubble. This bubble generates a pressure and shear spike which rupture the cell membrane. In this example, the lysing device (104) may be a thermal inkjet (TIJ) lysing device (104).

In another example, the lysing device (104) may be a piezoelectric device. As a voltage is applied, the piezoelectric device changes shape which generates a pressure pulse in the lysing chamber (122) that creates the shear which ruptures the cell membrane. In this example, the lysing device (104) may be a piezoelectric inkjet (PIJ) lysing device (104).

In yet another example, the lysing device (104) may be an electrostatic membrane or other mechanical actuator. While particular reference is made to particular lysing devices (104), any number of lysing devices (104) could be used in accordance with the principles described herein, a few examples of which have been provided above.

The lysis monitoring device (100) also includes at least one pair of electrodes (106) disposed within the lysing chamber (102). These electrodes (106) detect a level of conductivity within the lysing chamber (102). That is, incoming cells to a lysing chamber (102), and the solution in which they are contained, have a predetermined electrical conductivity. Any change to the contents of the lysing chamber (102) will effectively change the electrical conductivity within the lysing chamber (102). Specifically, as the cells are ruptured and the nucleic acid pours out, the conductivity would increase. To measure the conductivity, a resistance of solution between electrode (106) plates is measured and a conductivity determined therefrom. In some examples, a single pair of electrodes (106) are used, with one electrode plate placed at either end of a lysing chamber (102). In another example, multiple pair of electrodes (106) are used. For example, one pair of electrode (106) plates could be placed at the inlet and another pair of electrode (106) plates placed at the outlet. In other examples, a three electrode (106) or a four electrode (106) system could be used.

As described above, each cell releases approximately the same amount of ions during lysis. Accordingly, the electrodes (106) may be able to detect how many cells have been lysed based on a difference in the conductivity within the lysing chamber (102) before and after a lysis operation.

The lysis monitoring device (100) also includes a controller (108) to determine when the cell membrane has ruptured based on detected levels of conductivity in the lysing chamber (102). That is, the controller (108) may compare detected levels of conductivity within the lysing chamber (102) with a threshold level of conductivity associated with a ruptured cell. Accordingly, once the detected level of conductivity within the lysing chamber (102) has reached the threshold value, the controller (108) may determine that a cell has been ruptured.

While specific reference is made to cell rupture as the threshold, other thresholds may be used. For example, a less than entire cell rupture may be sufficient for some analytic purposes. In this example, the threshold conductivity relied on by the controller (108) may map to this desired level of cell rupture. Therefore, the controller (108) determines that a cell is “sufficiently lysed” when this threshold conductivity value has been reached, whatever that threshold conductivity may be. That is, a user may determine a satisfactory level of cell rupture for a particular chemical operation. That predetermined degree of cell rupture is mapped to a conductivity level expected when the degree of cell rupture is reached. Accordingly, during operation, the controller (108) determines that a cell has been satisfactorily lysed when it reaches that set threshold conductivity level that maps to any desired degree of cell rupture.

Accordingly, the present specification describes a lysis monitoring device (100) that monitors the lysis operation. Such control can provide a closed-loop feedback to ensure complete lysis. Moreover, such control can be used to control lysing parameters such as lysing intensity and lysis duration. The lysis monitoring device (100) having more control therein, enhances the efficiency of downstream analytics as subsequent systems can know with certainty an amount of starting material. Such knowledge increases the reliability and credibility of any final results/analysis.

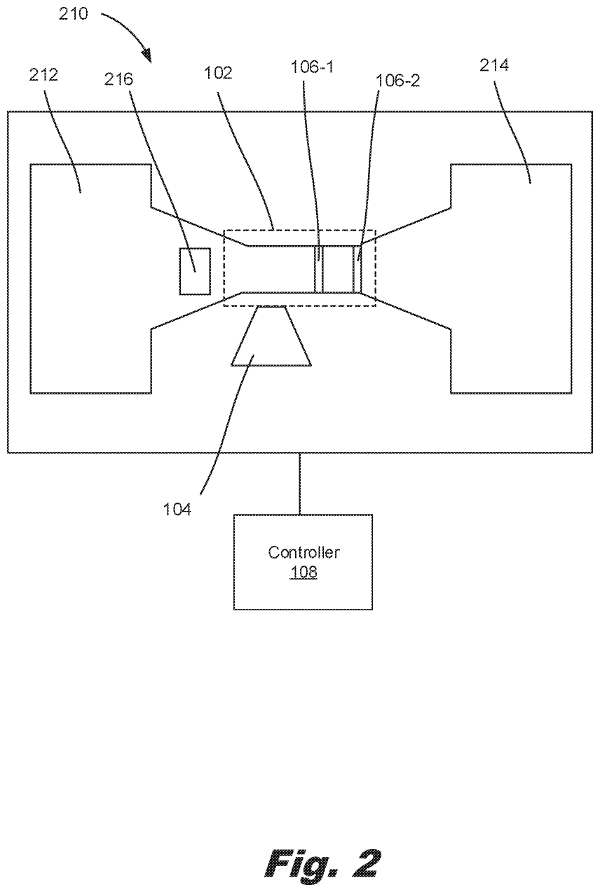

FIG. 2 is a diagram of a conductivity-based cell lysis system (210), according to an example of the principles described herein. They cell lysis system (210) includes a cell fluid inlet (212) to hold the cell fluid. The cell fluid inlet (212) stores fluid that contains cells to be analyzed. That is, the cell fluid includes various cells of interest that are to be lysed, such as cells cultured from plants, animals, or bacteria, which cells are suspended in an appropriate extracellular fluid medium such as interstitial fluid and blood plasma. For example, the cell fluid may include whole blood or components of blood including plasma in which red and white blood cells are suspended. In some examples, the cell fluid inlet (212) may be a reservoir where the cell fluid is contained or a channel through which the cell fluid is delivered.

The cell lysis system (210) also includes at least one lysis monitoring device (FIG. 1, 100). As described above, the lysis monitoring device (FIG. 1, 100) includes a lysing chamber (102) to receive the cell to be lysed. In some examples, such as that depicted in FIG. 2, the lysis chamber (102) has a reduced cross-section relative to the cell fluid inlet (212) and a reduced cross-section relative to the lysate outlet (214). The reduced cross-section of the lysing chamber (102) creates a constriction, which acts as a lysing element in some examples. That is, the reduced cross-section of the lysing chamber (102) increases pressure within the fluid, which can cause the cell membrane/wall to rupture.

As described above, the lysis monitoring device (FIG. 1, 100) also includes a lysing device (104). As described above in some examples, the lysing device (104) may reside within the lysing chamber (102). In other examples, such as that depicted in FIG. 2, the lysing device (104) may reside outside of the lysing chamber (102) yet produce a lysing effect within the lysing chamber (102). For example, the lysing device (104) may be an ultrasonic device that generates high energy sonic waves. These high energy waves may travel through the wall of the lysing chamber (102) to shear the cells disposed therein.

While particular reference is made to a particular lysing device (104) other lysing devices (104), some of which have been described above, may be implemented in accordance with the principles described herein.

FIG. 2 also clearly depicts the pair of electrodes (106-1, 106-2) that are disposed in the lysing chamber (102). As described above, these electrodes (106) may be metallic plates with a gap between them that can detect a resistance of a fluid in the gap. The measured resistance is indicative of a conductivity of the solution in the lysing chamber (102).

As depicted in FIG. 2, in some examples the lysing device (104) is disposed upstream of the electrodes (106). In this example, there may be a flow through the lysing chamber (102). That is, the fluid with the lysing chamber (102) may first flow past a lysing device (104) which lyses the cell. The solution then passes between the electrodes (106-1, 106-2) to determine if the cell has been sufficiently lysed, as defined by the threshold reference conductivity measure.

To generate such a flow, the lysis monitoring device (FIG. 1, 100) includes a main pump (216). That is, the main pump (216) directs fluid from the cell fluid inlet (212) through the lysing chamber (102) and past the lysing device (104) and electrodes (106). The main pump (216) may take many forms. For example, the main pump (216) may be integrated into a wall of the lysing chamber (102). More specifically, the main pump (216) may be a thermal resistor that is integrated into a wall of the lysing chamber (102). As used in the present specification, a wall refers to any wall of the chamber and may include a sidewall, a ceiling, and/or a floor.

In some examples, the main pump (216) is a TIJ pump. That is, as a firing resistor heats up, a portion of the fluid in the cell fluid inlet (212) vaporizes to generate a bubble. This bubble pushes fluid through the inlet (212) into the lysing chamber (102). In this example, the main pump (216) may be a TIJ pump.

In another example, the main pump (216) may be a piezoelectric device. As a voltage is applied, the piezoelectric device changes shape which generates a pressure pulse that pushes the fluid into the lysing chamber (102). In this example, the main pump (216) may be a PIJ pump. In yet another example, the main pump (216) may be an electrostatic membrane.

In any of these examples the energy applied through the main pump (216) may be less than the energy applied through a lysing device (104). Applying lesser energy through the main pump (216) ensures that the cell membrane/wall does not rupture as it passes the main pump (216).

Once fluid has passed by the electrodes (106) the lysed fluid is passed to a lysate fluid outlet (214) which holds the lysate until further processing. In some examples, the lysate fluid outlet (214) may be fluidly coupled to a downstream system for further analysis of the contents of the cell. In some examples, the lysate fluid inlet (212) may be a reservoir where the lysate fluid is contained or a channel through which the lysate fluid is delivered

FIG. 2 also depicts the controller (108). In some examples, the controller (108) includes a processor and memory. The controller (108) may additionally include other electronics (not shown) for communicating with and controlling the various components of cell lysis system (210), such as discrete electronic components and an ASIC (application specific integrated circuit). As a specific example, the controller (108) may control operation of the main pump (216) and other pumps during cell lysis. For example, if the lysis monitoring device (FIG. 1, 100) indicates that a cell has not ruptured or is otherwise not sufficiently lysed, the controller (108) may activate various combinations of pumps to return the cell to the lysing chamber (102) such that a second attempt at lysing can be carried out.

Also, as described above, the controller (108) may receive the measurements of the electrodes (106-1, 106-2) to determine if a cell has been lysed and/or determine how many cells have been lysed. That is, the electrodes (106) may provide a conductivity measurement. The controller (108) can compare this value to a threshold conductivity value that maps to a desired level of lysis. If the measured value is greater than the reference value, a determination made, and a count incremented, regarding cell lysis. By comparison, if the measured value is less than the reference value, the controller (108) may activate certain components to re-deploy the lysing device (104) in a second attempt to lyse the particular cell. Thus a controlled feedback for cell lysis is achieved based on monitoring the conductivity within a lysing chamber (102) where the lysing occurs.

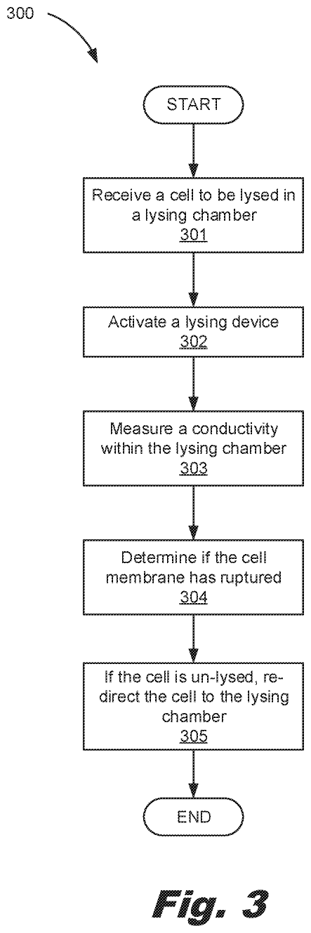

FIG. 3 is a flowchart of a method (300) for conductivity-based lysis monitoring, according to an example of the principles described herein. According to the method (300) a cell to be lysed is received (block 301) in a lysing chamber (FIG. 1, 102). That is, a fluid that contains a cell is transported into the lysing chamber (FIG. 1, 102). In some examples this is done by activating a main pump (FIG. 2, 216) which is integrated into a wall of the lysing chamber (FIG. 1, 102) or a cell fluid inlet (FIG. 2, 212). In other examples, this may be done by other mechanisms. The activation of the main pump (FIG. 2, 216) generates a fluid flow that transports the cell past a lysing device (FIG. 1, 104).

The lysing device (FIG. 1, 104) is activated (block 302) to carry out the desired lysing. Activating (block 302) the lysing device (FIG. 1, 104) may take many forms based on the type of lysing device (FIG. 1, 104) used. For example, it may include passing an electrical activation signal to the lysing device (FIG. 1, 104). The flow through the lysing chamber (FIG. 1, 102) causes the fluid and potentially lysed cell past the lysing device (FIG. 1, 104) and towards the electrode (FIG. 1,106).

Once between the electrodes (FIG. 1, 106) the conductivity can be measured (block 303). The measured value can be used to determine whether a cell wall/membrane has ruptured or not. Accordingly, the measured conductivity is analyzed (block 304) to determine if the cell membrane has ruptured. That is, the controller (FIG. 1, 108) can consult a database with a mapping to determine if the measured level of conductivity is lesser or greater than a value indicative of a threshold lysing. If greater than the threshold value, the controller (FIG. 1, 108) may activate other components such as a pump to direct the lysate to a subsequent analytic chamber. If not greater than the threshold value, the controller (FIG. 1, 108) may activate other components, such as a return pump, to re-direct (block 305) the un-lysed cell back to the lysing chamber (FIG. 1, 102) for additional operation by the lysing device (FIG. 1, 104) to hopefully lyse the cell in a second attempt.

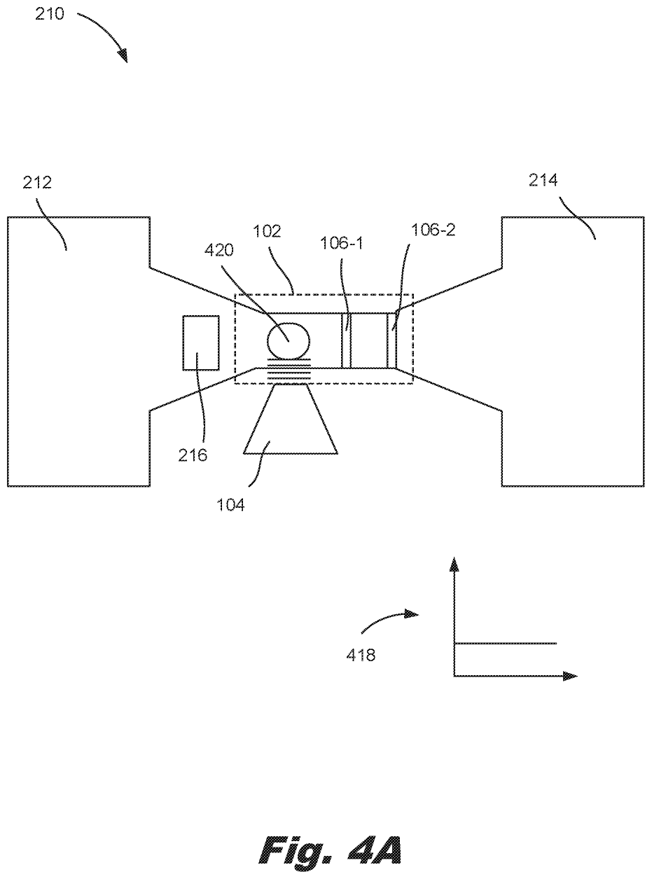

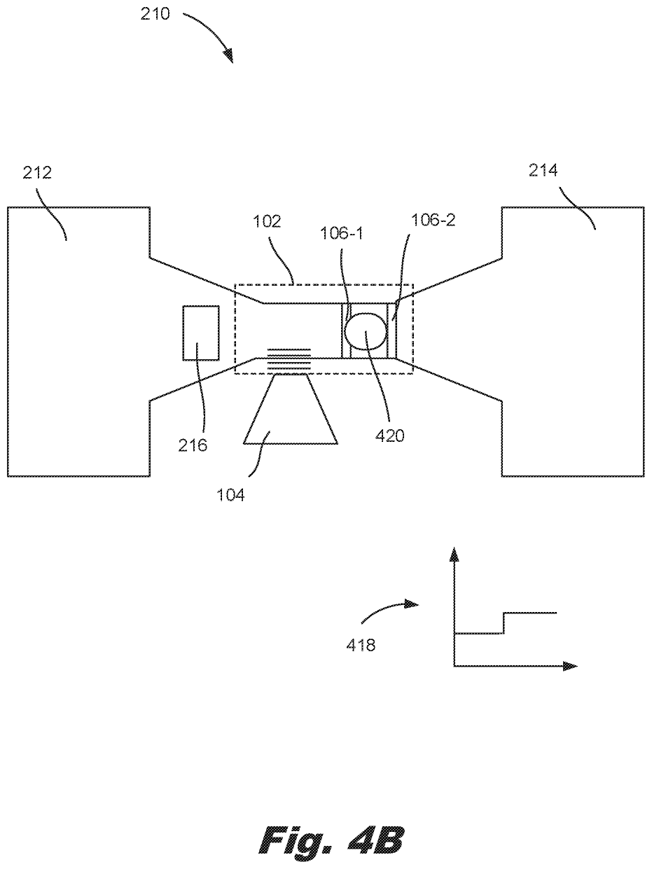

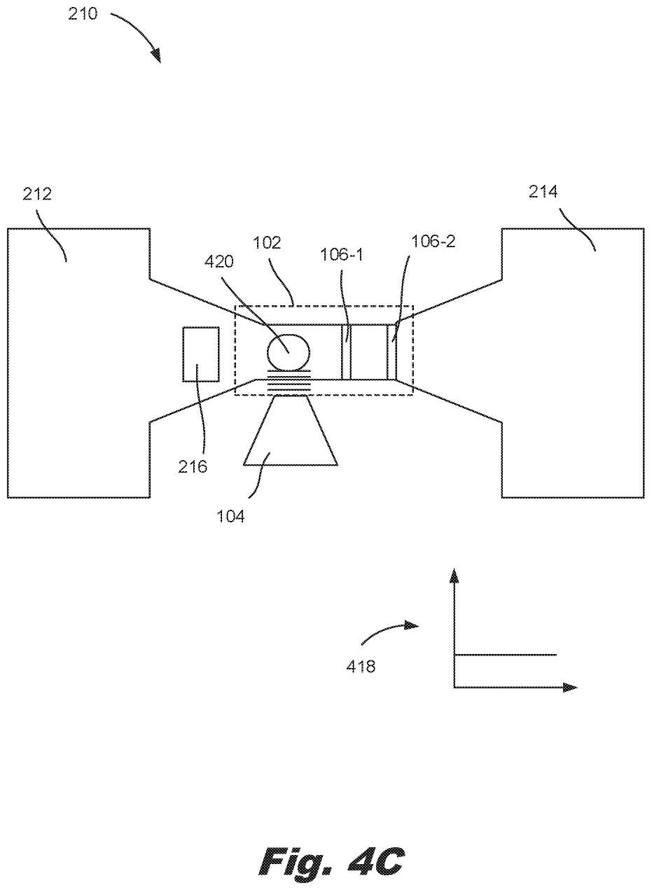

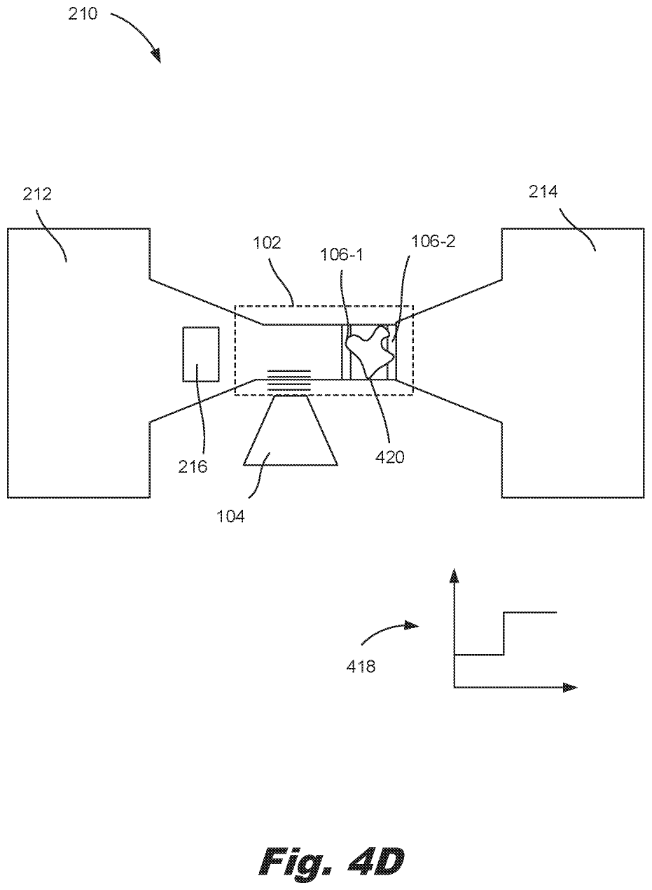

FIGS. 4A-4D are diagrams of conductivity-based lysis monitoring, according to an example of the principles described herein. As described above, the cell lysis system (210) may provide for closed-loop feedback to ensure an appropriate lysis yield. For example, as depicted in FIG. 4A, a cell (420) to be lysed is passed from a cell fluid inlet (212) to a lysing chamber (102) via action of a main pump (216). As no lysing has yet occurred, the conductivity within the lysing chamber (102) as measured by the electrodes (106-1, 106-2) is constant as indicated by the graph (418). The lysing device (104), being triggered by a detected presence of the cell (420) or being continuously operated, performs a lysing operation on the cell (420).

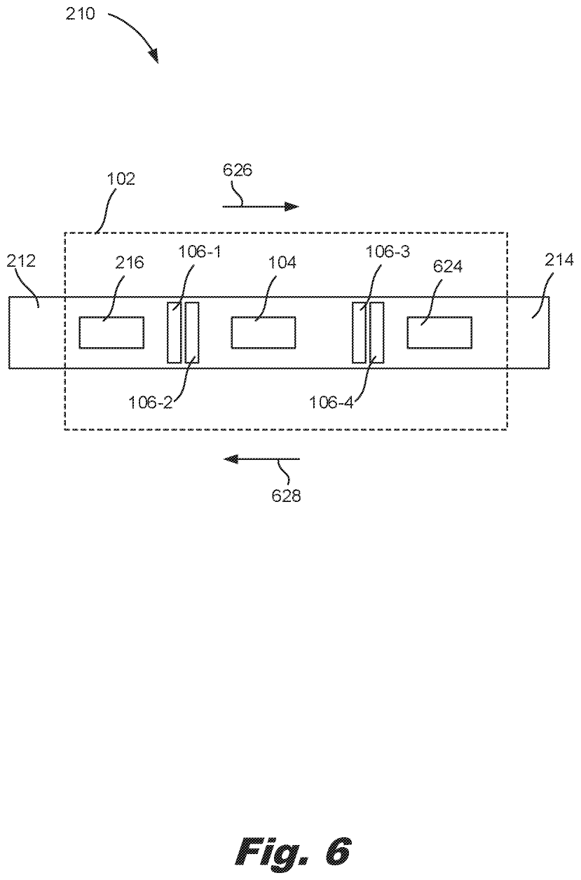

In the example depicted in FIGS. 4A-4D, the lysing device (104) for whatever reason has failed to rupture the cell (420) to a desired amount. However, the mere presence of the cell (420) between the electrodes (106) as indicated in FIG. 4B may cause a smaller change in the conductivity within the lysing chamber (102) as indicated by the graph (418). In some cases, the presence of the cell (420) between the electrodes can even cause a decrease in conductivity due to the low conductivity of the cell membrane. Accordingly, an output of the electrodes (106-1, 106-2) would indicate a slight change in conductivity within the lysing chamber (102) as indicated by the graph (418) in FIG. 4B, this change may in some examples be used to trigger the activation of the lysing device (104) as described below in FIG. 6.

Accordingly, the controller (FIG. 1, 108) may activate certain components such as a return pump and/or the main pump (216) to place the cell (420) adjacent with the lysing device (104) again as depicted in FIG. 4C in an attempt to re-lyse the cell (420).

In this example, the lysing device (104) successfully lyses the cell (420) as indicated in FIG. 4D. The lysed cell (422) changes the conductivity within the lysing chamber (102). The change in conductivity is detected by the electrodes (106), which change is illustrated in the graph (418). Accordingly, the cell lysis system (210) provides a feedback control loop wherein cell lysis can be ensured. That is, based on feedback from the electrodes (106) lysis is either continued or stopped. By tracking the number of cells (420) and the conditions to lyse them, information about cell population in the sample can be obtained.

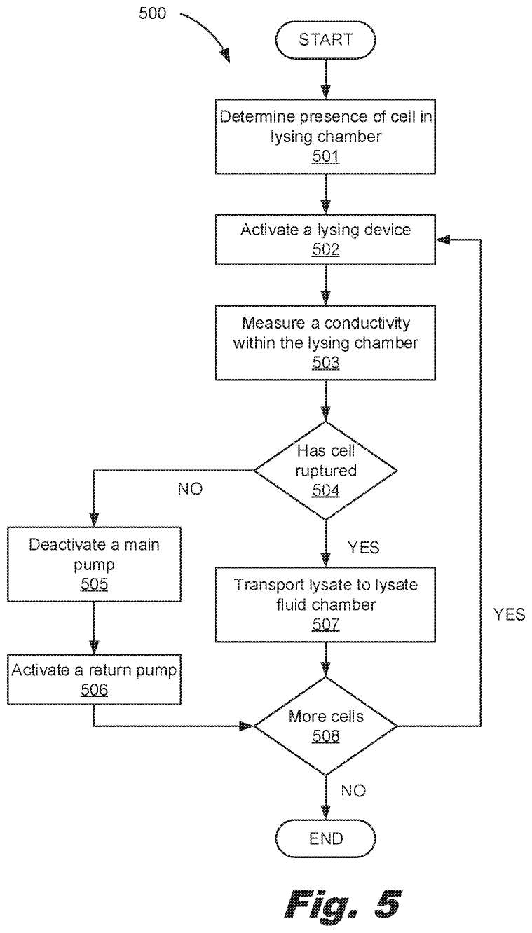

FIG. 5 is a flowchart of a method (500) for conductivity-based lysis monitoring, according to another example of the principles described herein. In some examples, the lysing device (FIG. 1, 104) may be triggered by the presence of a cell (FIG. 4, 420) within the lysing chamber (FIG. 1, 102). That is, the lysing device (FIG. 1, 104) is inactive when a cell (FIG. 4, 420) is not found within the lysing chamber (FIG. 1, 102) and upon the reception of a cell (FIG. 4, 420) in the lysing chamber (FIG. 1, 102), the lysing device (FIG. 1, 104), and potentially other components such as the electrodes (FIG. 1, 106), are activated. Accordingly, in this example, the method (500) includes determining (block 501) a presence of a cell (FIG. 4, 420) to be lysed in the lysing chamber (FIG. 1, 102). When such a cell (FIG. 4, 420) presence is determined (block 502) the lysing device (FIG. 1, 104) and other components may be activated. This example, may conserve resources as the lysing device (FIG. 1, 104) and electrodes (FIG. 1, 106) are just powered when needed. Doing so may also increase the longevity of these components.

In another example, the lysing device (FIG. 1, 104) is active regardless of a presence of a cell (FIG. 4, 420) to be lysed within the lysing chamber (FIG. 1, 102). Doing so may simplify the operation of the cell lysis system (FIG. 2, 210) as no complex components are used to shut down and start up the lysing device (FIG. 1, 104) and electrodes (FIG. 1, 106). As with determining whether a cell (FIG. 4, 420) has sufficiently lysed, the presence of a cell (FIG. 4, 420) may be determined (block 501) relying on conductivity measurements. That is, in addition to the lysing of a cell (FIG. 4, 420) changing the conductivity within the lysing chamber (FIG. 1, 102), the mere presence of a cell (FIG. 4, 420) changes the conductivity within the lysing chamber (FIG. 1, 102). Accordingly, the electrodes (FIG. 1, 106) may detect a cell (FIG. 4, 420) in the lysing chamber (FIG. 1, 102) by detecting a change to the conductivity therein. Accordingly, the controller (FIG. 1, 108) receives the measurements and determines (block 501) that a cell (FIG. 4, 420) is present.

In either case, the lysing device (FIG. 1, 104) is activated (block 502) and a conductivity within the lysing chamber (FIG. 1, 102) is measured (block 503). These operations may be performed as described above in connection with FIG. 3. It is then determined (block 504) whether the cell (FIG. 4, 420) membrane has ruptured. This may be done by analyzing measurements collected by the electrodes (FIG. 1, 106) as described above in connection with FIG. 3. If the cell (FIG. 4, 420) has ruptured (block 504, determination YES), the lysate may be transported (block 507) to the lysate fluid outlet (FIG. 2, 214) and/or to a downstream analysis chamber.

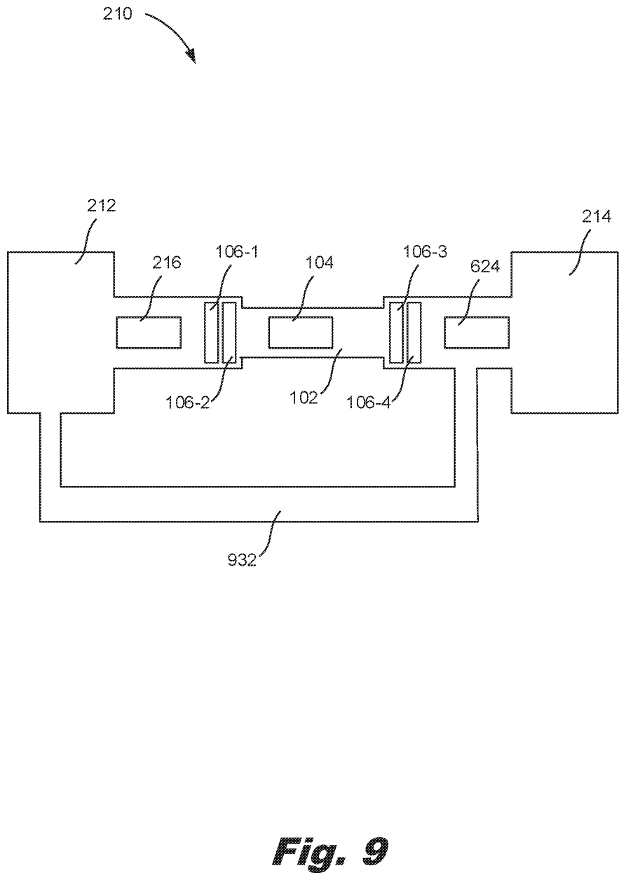

By comparison, if the cell (FIG. 4, 420) has not ruptured (block 504), determination NO), a main pump (FIG. 2, 216) may be deactivated (block 505) and a return pump may be activated (block 506) to re-direct the cell (FIG. 4, 420) to the lysing chamber (FIG. 1, 102). That is, to ensure proper lysing, the un-lysed cell (FIG. 4, 420) is returned to the lysing chamber (FIG. 1, 102). However, operation of the main pump (FIG. 2, 216) generates a flow contrary to the passage of the cell (FIG. 4, 420) back to the lysing device (FIG. 1, 104). That is, the re-directed flow is contrary to the flow established by the main pump (FIG. 2, 216). Accordingly, the main pump (FIG. 2, 216) is de-activated (block 505) and a return pump is activated (block 506) such that the un-lysed cell (FIG. 4, 420) can be re-lysed. In either case, it is determined (block 508) if another, or more cells (FIG. 4, 420) are present in the lysing chamber (FIG. 1, 102). If not, (block 508, determination NO), the process ends. If so, (block 508, determination YES), the lysing device (FIG. 1, 104) is again activated (block 502) and the operation continues as described above until all cells in a sample have been lysed. Examples of such de-activation and activation are presented in connection with FIGS. 6 and 9.

FIG. 6 is a diagram of a conductivity-based cell lysis system (210), according to another example of the principles described herein. Specifically, in this example, the cell lysis system (210) includes the cell fluid inlet (212) and lysate fluid outlet (214) as described above. The cell lysis system (210) also includes the lysis monitoring device (FIG. 1, 100) with its lysing chamber (102) and electrodes (106). For simplicity in this example, the controller (FIG. 1, 108) is not depicted.

As described above, in some examples, the lysing chamber (102) may have a reduced cross section. However, in other examples such as depicted in FIG. 6, the lysing chamber (102) may have a same cross-section as the cell fluid inlet (212) and the lysate outlet (214).

As depicted in FIG. 6, in some examples the lysing device (104) may be disposed between pairs of electrodes (106). That is, in this example multiple pairs of electrodes (106) are used to determine a conductivity within the lysing chamber (102). For example, a first pair of electrodes (106-1, 106-2) may be disposed near an inlet of the lysing chamber (102) and a second pair of electrodes (106-3, 106-4) may be disposed near an outlet of the lysing chamber (102). In this example, the electrode (106) pairs can be used to determine a conductivity within the entirety of the lysing chamber (102) rather than just a portion. By covering the entirety of the lysing chamber (102), this configuration is also effective at determining the presence of a cell (FIG. 4, 420) anywhere in the lysing chamber (102).

In this example, each pair of electrodes (106) may determine a conductivity and a difference used to determine lysing and/or cell presence. That is, a first pair of electrodes (106-1, 106-2) determine a conductivity at an inlet and a second pair of electrodes (106-3, 106-4) determine a conductivity at an outlet. In addition, the two can then be compared and if a difference between the two is a threshold amount, it may be determined that a cell (FIG. 4, 420) is present and/or whether that cell (FIG. 4, 420) has been sufficiently lysed as defined by the threshold value set by a user. In some examples sufficiently lysed may indicate that a cell (FIG. 4, 420) has ruptured and contents have spilled out of the cell (FIG. 4, 420).

The first pair of electrodes (106-1, 106-2) can also be used to determine the presence of a cell (FIG. 4, 420) in the lysing chamber (102) and to control a main pump (216) and lysing device (104). That is, as described above a change in conductivity can indicate that a cell (FIG. 4, 420) has ruptured and can also indicate that a cell (FIG. 4, 420) is present. In this example, the main pump (216) may be activated to generate a flow and move a cell (FIG. 4, 420) into a lysing chamber (102). In this example, the first pair of electrodes (106-1, 106-2) may indicate a change in conductivity that maps to a cell presence. In this example, the controller (FIG. 1, 108) shuts off the main pump (216) to stop the flow such that the cell (FIG. 4, 420) is maintained in the lysing chamber (102). The controller (FIG. 1, 108) may also activate the lysing device (104) to begin the lysing process.

FIG. 6 also depicts the main pump (216) and a return pump (624) that may be used to direct flow in a desired direction. As with the main pump (216), the return pump (624), may be a TIJ pump, a PIJ pump, an electrostatic membrane or other mechanical actuator. The main pump (216), upon activation may direct a cell (FIG. 4, 420) through the lysing chamber (102) as indicated by the arrow (626), that is, past the lysing device (104). When a sensed conductivity indicates that the cell (FIG. 4, 420) has not ruptured, the cell lysis system (210) may operate to return the cell (FIG. 4, 420) to the lysing chamber (102) for a second attempt. In this example, the controller (FIG. 1, 108) de-activates the main pump (216) so as to stop the flow in the direction indicated by the arrow (626). The controller (FIG. 1, 108) then activates the return pump (624) such that a flow is generated in the direction indicated by the arrow (628) such that the cell (FIG. 4, 420) may be re-lysed. That is, the return pump (624) re-directs the cell (FIG. 4, 420) to the lysing chamber (102) through an outlet of the lysing chamber (102). In this example, the first pair of electrodes (106-1, 106-2) again detects the presence of the cell (FIG. 4, 420) in the lysing chamber (102) and stops the return pump (624) and activates the main pump (216) and/or lysing device (104) in a second attempt lysing operation. In some examples, such a feedback operation may be performed automatically such that a closed loop feedback may be exhibited without user involvement.

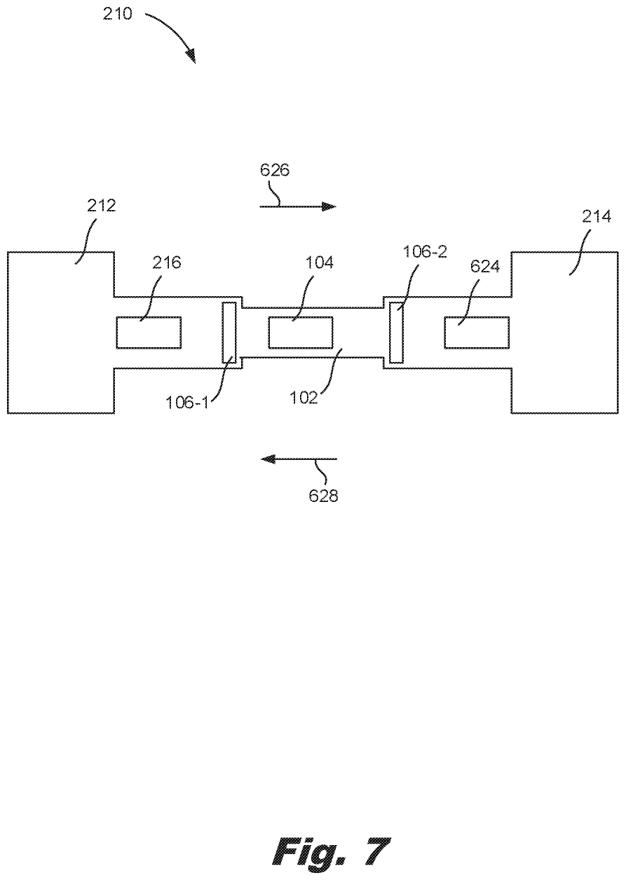

FIG. 7 is a diagram of a conductivity-based cell lysis system (210), according to another example of the principles described herein. In this example, rather than having multiple pairs of electrodes (106), the lysis monitoring device (FIG. 1, 100) includes a single pair of electrodes (106-1, 106-2), with a plate disposed on either side of the lysing chamber (102). In this example, rather than measuring a conductivity at an inlet and an outlet and comparing the differences, a single value is measured which is indicative of a conductivity within the entirety of the lysing chamber (102).

In some examples, multiple lysing devices (104) may be present. FIG. 7 depicts such an example. That is, as depicted in FIG. 7 one lysing device (104) may be a thermal resistor disposed within the lysing chamber (102). In this example, additional lysing may be effectuated by the narrowed channel, i.e., the constriction between the cell fluid inlet (212) and the lysing chamber (102).

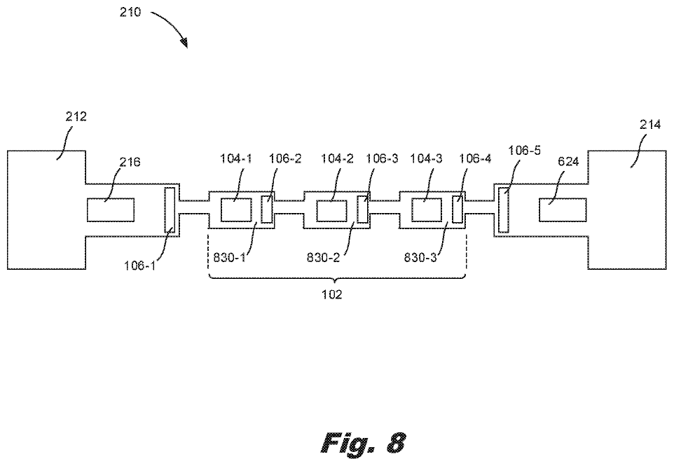

FIG. 8 is a diagram of a conductivity-based cell lysis system (210), according to another example of the principles described herein. In the example depicted in FIG. 8, the lysing chamber (102) includes multiple sub-chambers (830-1, 830-2, 830-3). In this example, each lysing sub-chamber (830) may or may not have its own lysing device (104-1, 104-2, 104-3) disposed therein. That is, as in the example depicted in FIG. 8, each sub-chamber (830) may have a unique lysing device (104). However, in other examples, a subset of the sub-chambers (830) may have a lysing device (104) disposed therein.

Also in this example, each sub-chamber (830) may have at least one electrode (106). The electrodes (106) in the different sub-chambers (830) may be paired with one another to give a conductivity measure to that point. For example, a first electrode (106-1) and a second electrode (106-2) may be paired to determine a conductivity in the first sub-chamber (830-1). In another example, the third electrode (106-3) may be paired with the second electrode (106-2) to determine a conductivity in the second sub-chamber (830-2).

While FIG. 8 depicts a single electrode (106) within each sub-chamber (830) in other examples, additional electrodes (106) may be used. For example, differential pairs of electrodes (106) may be disposed within each sub-chamber (830). In some examples, a lysing device (104) in a downstream sub-chamber (830) is activated when it is determined that a cell (FIG. 4, 420) membrane has not been ruptured, or otherwise insufficiently lysed, in an upstream sub-chamber (830). For example, a cell (FIG. 4, 420) in the first sub-chamber (830-1) may not rupture as indicated by corresponding electrode (106) measurements. Accordingly, as the cell (FIG. 4, 420) passes to the second sub-chamber (830-2), the second lysing device (104-2) may be activated. If the second lysing device (104-2) lyses the cell (FIG. 4, 420), subsequent lysing devices may be deactivated and the fluid passed to the lysate fluid outlet (214) without further activation of the subsequent lysing devices (104). Thus, a refined and sophisticated real time monitoring of cell lysing is provided which also ensures that cell lysis occurs as desired.

FIG. 9 is a diagram of a conductivity-based cell lysis system (210), according to another example of the principles described herein. In the example depicted in FIG. 9, the cell lysis system (210) further includes a return channel (932) through which the return pump (624) re-directs fluid to the lysing chamber (102). That is, rather than re-directing an un-lysed cell against the flow generated by the main pump (216), fluid is directed through the return channel (932). In this example, the main pump (216) is not de-activated when an un-lysed cell is detected. Rather, it is maintained active to help draw fluid through the return channel (932) to the lysing chamber (102).

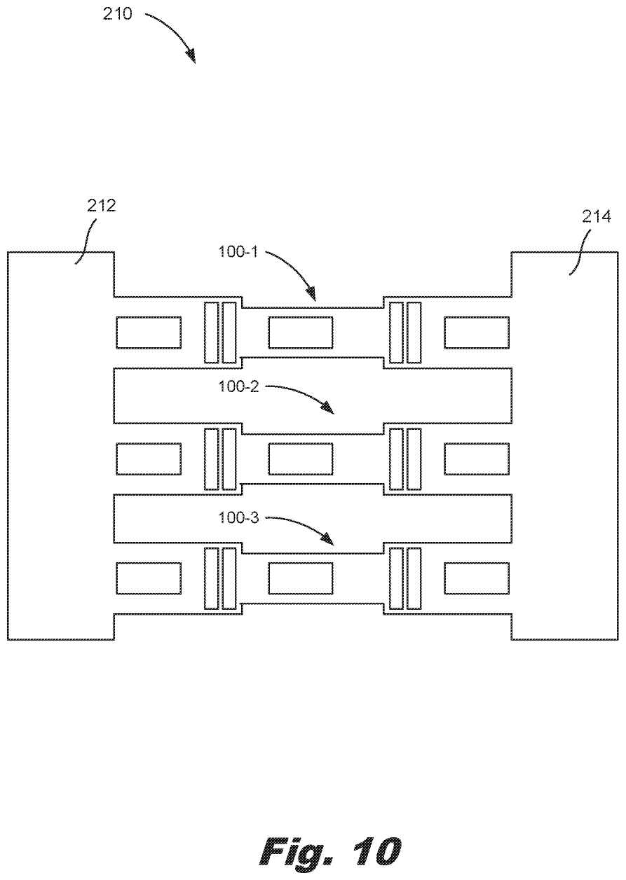

FIG. 10 is a diagram of a conductivity-based cell lysis system (210), according to another example of the principles described herein. In the example depicted in FIG. 10, the cell lysis system (210) includes multiple lysis monitoring devices (100-1, 100-2, 100-3). For simplicity, the components that make up the lysis monitoring devices (100-1, 100-2, 100-3) are not indicated with reference numbers. In this example, the multiple lysis monitoring devices (100) allow for the parallel processing of a sample fluid thus increasing the overall efficiency of the cell lysis system (210).

In summary, using such a lysis system 1) provides for effective monitoring of cell lysis; 2) ensures sufficient lysis without degradation to cell contents; 3) provides control of the amount of analyte to be delivered downstream; 4) identifies subsets of cell population that are difficult to lyse; and 5) provides a feedback signal for automated control of the lysis operation. However, the devices disclosed herein may address other matters and deficiencies in a number of technical areas.

What is claimed is:

1. A lysis monitoring device, comprising:
a lysing chamber to receive a cell to be lysed;
at least one lysing device to rupture a cell membrane;
at least one pair of electrodes disposed in the lysing chamber to detect a level of conductivity in the lysing chamber; and
a controller to determine when the cell membrane has ruptured based on detected levels of conductivity in the lysing chamber.

2. The device of claim 1, wherein the lysing device is disposed between the at least one pair of electrodes.

3. The device of claim 1, wherein:
the cell flows through the lysing chamber during lysing; and
the lysing device is disposed upstream of the pair of electrodes in a flow.

4. The device of claim 1, wherein:
the lysing chamber comprises multiple sub-chambers;
at least one sub-chamber has a corresponding lysing device; and
at least one sub-chamber has a corresponding electrode to detect a level of conductivity in the sub-chamber.

5. The device of claim 4, wherein a lysing device of a downstream sub-chamber is activated when it is determined that the cell membrane is not sufficiently lysed in an upstream sub-chamber.

6. The device of claim 1, further comprising:
a main pump to generate a fluid flow through the lysing chamber; and
a return pump to re-direct the cell to the lysing chamber when the cell membrane is not sufficiently lysed following lysing.

7. The device of claim 6, wherein the return pump re-directs the cell to the lysing chamber through at least one of:
an outlet of the lysing chamber; and
a return channel.

8. The device of claim 1, wherein the pair of electrodes is selected from the group consisting of:
a single pair of electrodes; and
two pair of electrodes, each pair space apart from the other.

9. A cell lysis system, comprising:
a cell fluid inlet;
at least one lysis monitoring device fluidly connected to the cell fluid inlet, each lysis monitoring device comprising:
a lysing chamber to receive a cell to be lysed;
a lysing device disposed at least partially in the lysing chamber to rupture a cell membrane;
a pair of electrodes disposed in the lysing chamber to detect a level of conductivity in the lysing chamber;
a pump to generate a fluid flow through the lysing chamber; and
a controller to perform at least one of:
determine a presence of the cell to be lysed in the lysing chamber;
activate the lysing device in response to a determined presence of the cell; and
determine when the cell membrane has ruptured based on detected levels of conductivity in the lysing chamber; and
a lysate outlet to pass a lysate from the at least one lysis monitoring device.

10. The system of claim 9, wherein the lysing chamber has a reduced cross-section relative to an inlet and outlet of the lysing chamber.

11. The system of claim 9, wherein the at least one lysis monitoring device comprises multiple lysis monitoring devices to operate in parallel.

12. A method, comprising:
receiving, in a lysing chamber, a cell to be lysed;
activating a lysing device in the lysing chamber which is to rupture a cell membrane of the cell in the lysing chamber;
measuring a conductivity within the lysing chamber;
analyzing the conductivity within the lysing chamber to determine that the cell membrane has ruptured; and
if the cell is un-lysed, re-directing the cell to the lysing chamber.

13. The method of claim 12, further comprising determining a presence of the cell to be lysed in the lysing chamber, and wherein the lysing device is activated in response to a determined presence of the cell.

14. The method of claim 12, wherein the lysing device is active regardless of the presence of the cell to be lysed in the lysing chamber.

15. The method of claim 12 further comprising, if the cell is un-lysed:
deactivating a main pump which draws a cell fluid through the lysing chamber; and
activating a return pump to re-direct the cell to the lysing chamber.
